(19) United States
(12) Patent Application Publication
(10) Pub. No.: US 2013/0237974 A1
(43) Pub. Date: Sep. 12, 2013

(54) CATHETER APPARATUS AND METHOD

(71) Applicants: Alexander Cornelis Elisabeth Van Thoor, Utrecht (NL); David Stecher, Utrecht (NL); Diederick Marco Keizer, Utrecht (NL)

(72) Inventors: Alexander Cornelis Elisabeth Van Thoor, Utrecht (NL); David Stecher, Utrecht (NL); Diederick Marco Keizer, Utrecht (NL)

(21) Appl. No.: 13/413,865

(22) Filed: Mar. 7, 2012

(51) Int. Cl. A61B 18/24; A61B 18/20; A61B 17/32

(52) U.S. Cl. 606/15; 606/170; 606/14

(57) ABSTRACT

In some examples, a catheter apparatus, configured to cut through a vessel wall, includes an elongate body including a distal end. A cutting device, configured to selectively cut a hole in the vessel wall, is disposed at the distal end of the elongate body. The cutting device includes a substantially ring-shaped cutting portion including a first dimension and a second dimension, wherein the first dimension is greater than the second dimension. The gripping device, disposed at the distal end of the elongate body, is configured to grip the vessel wall to maintain the cutting device proximate the vessel wall during cutting of the hole in the vessel wall. The gripping device is further configured to retain a portion of the vessel wall removed during cutting of the vessel wall. In some examples, a method includes using a catheter apparatus to cut through a vessel wall.

### TECHNICAL FIELD

Various embodiments described herein relate to apparatuses, systems, and methods associated with anastomosis.

### BACKGROUND

Anastomosis, in the medical field, is a connection between two channels, for instance, blood vessels. Such connections can be made between sides of respective vessels, between ends of respective vessels, or between an end of a vessel and a side of another vessel. In various anastomosis procedures, a hole is formed in one or both of the vessels. Such anastomosis procedures can be used in various surgical procedures, such as, for instance, bypass procedures.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a perspective view of a distal end of an example catheter apparatus according to an embodiment of the invention.

FIG. 2 shows a side view of an example catheter apparatus according to an embodiment of the invention.

FIG. 3 shows a perspective view of a distal end of an example catheter apparatus according to an embodiment of the invention.

FIG. 4 shows an end view of a distal end of an example catheter apparatus according to an embodiment of the invention.

FIG. 5 shows an end view of a distal end of an example catheter apparatus according to an embodiment of the invention.

FIG. 6 shows a cross-sectional view of an example catheter apparatus according to an embodiment of the invention.

FIGS. 7A-7D show simplified cross-sectional views of an example catheter, according to an embodiment of the invention, being used to cut through a vessel wall.

FIG. 8A shows a hole cut through a vessel wall using the catheter apparatus of FIG. 4.

FIG. 8B shows a hole cut through a vessel wall using the catheter apparatus of FIG. 5.

### DETAILED DESCRIPTION

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

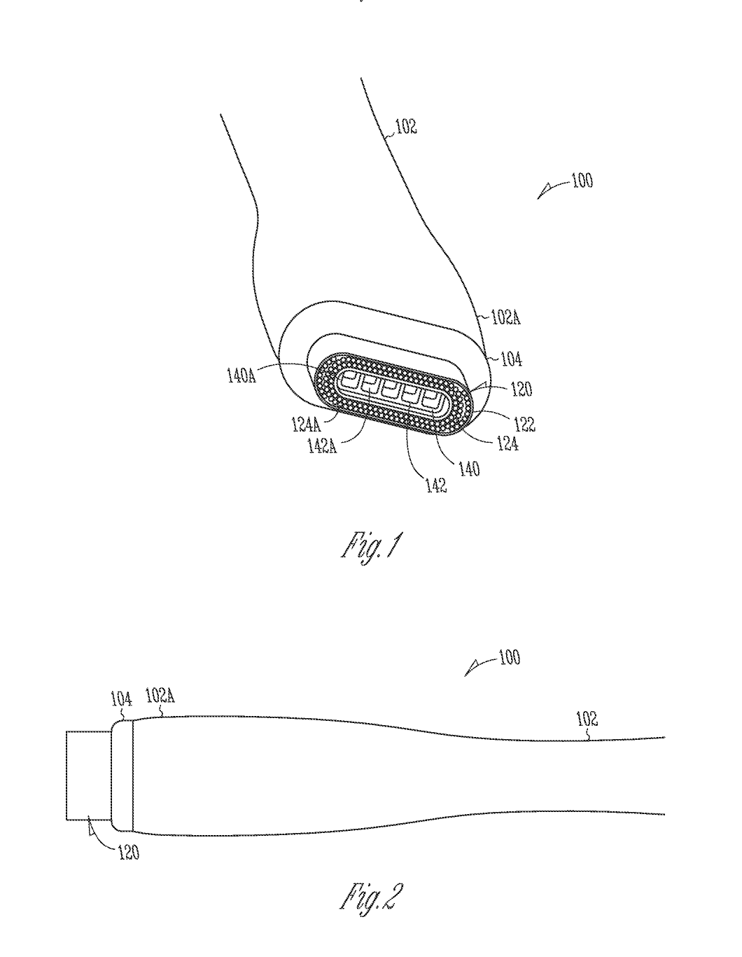

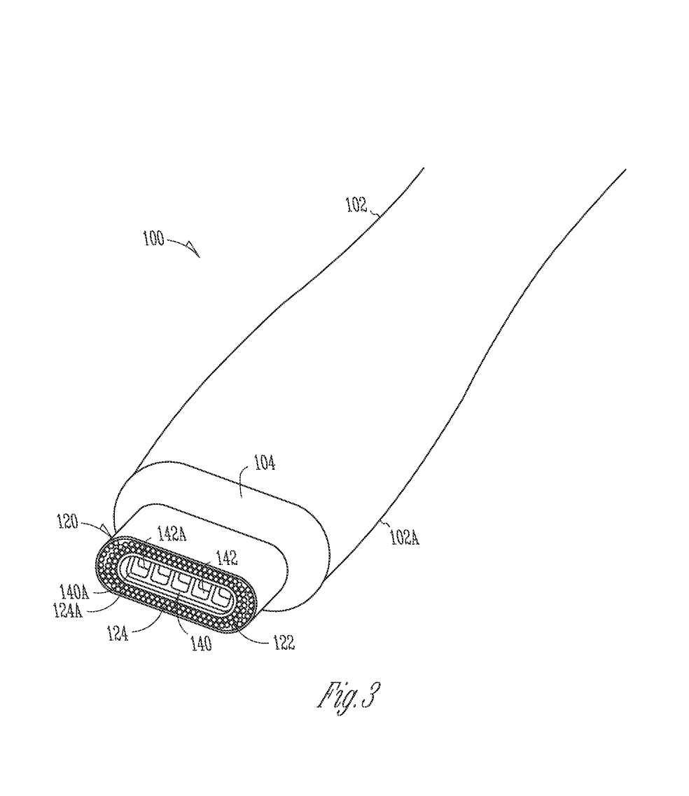

Referring to FIGS. 1-4, in some examples, a catheter apparatus 100 is configured to cut through a vessel wall. The catheter apparatus 100 can be used in various procedures including, but not limited to, anastomosis procedures in which two vessels are fluidly coupled to one another. In some examples, a hole can be cut in the side of one of the vessels (for instance, with end-to-side anastomosis techniques) or in the sides of both of the vessels (for instance, in side-to-side anastomosis techniques). Such anastomosis procedures can be used in various bypass procedures. In some examples, such anastomosis procedures can be used to join blood vessels in various areas of the body, including, but not limited to, in and around the brain, the heart, and the kidneys. Such anastomosis procedures can also be used for joining two tubular structures other than blood vessels, such as intestines, for instance.

In some examples, the catheter apparatus 100 includes an elongate body 102. In some examples, the elongate body 102 is configured for use in surgery. That is, the elongate body 102 can be formed from one or more biocompatible materials that can be relatively easily sterilized. In addition, the elongate body 102 can be formed from one or more flexible materials. In some examples, the elongate body 102 includes a tubular body. The elongate body 102 can include a distal end 102A configured for insertion within the body. In some examples, the distal end includes a widened portion 104 at or near the distal end 102A. In some examples, the widened portion 104 is sized to be at least as wide as the width (or diameter) of the vessel through which the catheter apparatus 100 is intended to be guided. By sizing the widened portion 104 at least as wide as the width of the vessel, the widened portion 104 can function to center the distal end 102A of the catheter apparatus 100 within the vessel to aid in guiding the catheter apparatus 100 through the vessel and positioning it for cutting of the vessel wall. In other examples, the widened portion 104 can be sized to be smaller than the width (or diameter) of the vessel through which the catheter 100 is intended to be guided to allow for greater ease of insertion and guiding of the catheter apparatus 100 within the vessel than if the widened portion 104 were sized as wide or wider than the vessel. In some examples, as described in greater detail herein, the widened portion 104 is sized to correspond with a ring, a clip, or another device used in an anastomosis procedure to inhibit the catheter apparatus 100 from being inserted too far into the vessel to lessen the likelihood of potentially accidentally cutting a portion of a vessel that was not intended to be cut. Although shown as separate from the elongate body 102, in some examples, the widened portion 104 can be integrally formed with the elongate body 102.

In some examples, the catheter apparatus 100 includes a cutting device 120 disposed at the distal end 102A of the elongate body 102. In some examples, the cutting device 120 extends beyond the distal end 102A. In other examples, the cutting device 120 can be flush with or recessed within the distal end 102A. In various examples, the cutting device 120 can extend beyond, be flush with, or be recessed within the widened portion 104. The cutting device 120, in some examples, includes a substantially ring-shaped cutting portion 122 configured to selectively cut a hole in the vessel wall. In some examples, the ring-shaped cutting portion 122 includes a first dimension and a second dimension, wherein the first dimension is greater than the second dimension.

In some examples, the widened portion 104 of the catheter apparatus 100 is sized to correspond with a ring, a clip, or another device used in an anastomosis procedure to inhibit the catheter apparatus 100 from being inserted too far into the vessel to lessen the likelihood of potentially accidentally cutting through a portion of a vessel that was not intended to be cut through. In examples in which the cutting device 120 extends beyond the distal end 102A, the cutting device 120 or the ring, clip, or other device can be sized to allow the cutting device 120 to fit through the ring, clip, or other device while the widened portion 104 can be sized to be larger than the ring, clip, or other device such that the catheter device 100 is inhibited from passing through the ring, clip, or other device, thereby inhibiting the catheter apparatus 100 from being inserted too far into the vessel to lessen the likelihood of potentially accidentally cutting something (such as a portion of a vessel) that was not intended to be cut. In examples in which the cutting device 120 is flush with or recessed within the distal end 102A, the widened portion 104 being sized to be larger than the ring, clip, or other device functions in a similar manner to inhibit the catheter device 100 from passing through the ring, clip, or other device to lessen the likelihood of potentially accidentally cutting something (such as a portion of a vessel) that was not intended to be cut. If the catheter apparatus 100 were able to be passed through the ring, clip, or other device, the cutting device 120 could potentially be placed in proximity of another structure (such as another portion of the vessel being cut or a portion of another vessel) and could potentially at least partially cut or damage the structure.

In some examples, the catheter apparatus 100 includes a gripping device 140 disposed at the distal end 102A of the elongate body 102. The gripping device 140 can be configured to grip the vessel wall to maintain the cutting device 120 proximate the vessel wall during cutting of the hole in the vessel wall. In some examples, the gripping device 140 can be disposed within and surrounded by the substantially ring-shaped cutting portion 122. In further examples, the gripping device 140 encompasses substantially the entire area within the substantially ring-shaped cutting portion 122. In other examples, the gripping device 140 can be disposed outside the substantially ring-shaped cutting portion 122. In still further examples, the gripping device 140 can be disposed both within and outside of the substantially ring-shaped cutting portion 122. The gripping device 140, in various examples, is configured to retain a portion of the vessel wall removed during cutting of the vessel wall. That is, with the substantially ring-shaped cutting portion 122, the cutting device 120 cuts or ablates a similarly sized and shaped ring-shaped portion from the vessel wall, which leaves a free portion of the vessel wall (substantially corresponding to the size and shape of the interior of the substantially ring-shaped cutting portion 122) now unattached to the vessel wall, which the gripping device 140 can retain after cutting of the blood vessel and remove from within the body of the patient.

In some examples, the gripping device 140 includes a vacuum gripping device 140 that creates a suction force to grip and retain the portion of the vessel wall. In some examples, the gripping device 140 can include a cover 142 at a distal end 140A of the gripping device 140 to inhibit unwanted objects from being sucked into the end of the gripping device 140. For instance, in some examples, the cover 142 can be configured to inhibit the cut-out portion of the vessel wall from being sucked into the gripping device 140. In some examples, the cover 142 can be configured to hold the vessel wall in a particular position with respect to the cutting device 120 during cutting of the vessel wall. For instance, maintaining the vessel wall in the particular position can, among other things, facilitate proper dimensioning of the hole to be cut by inhibiting the vessel wall from being sucked too far into the gripping device 140 (and potentially making a larger than intended hole in the vessel wall) or moving excessively during cutting (and potentially leading to a hole being formed with improper dimensions).

The cover 142, in some examples, can include one or more holes 142A to allow the suction force of the gripping device 140 to grip the portion of the vessel wall while, at the same time, inhibiting the portion of the vessel wall (or other objects larger than the one or more holes 142A in the cover 142) from being sucked into the distal end 140A of the gripping portion 140. The cover 142 shown in FIGS. 1 and 3 includes a grid-like pattern of substantially square-shaped holes 142A. However, in other examples, the cover can include differently configured holes (circular, for instance), a screen, protrusions or abutments extending within the distal end 140A, or other structures or configurations capable of inhibiting objects from being taken in by the gripping device 140. In some examples, the gripping device 140 is recessed with respect to the ring-shaped cutting portion 122, such that, when the gripping device 140 grips the vessel wall, a portion of the vessel wall is drawn within the recess (for instance, to abut the cover 142). Such a configuration allows for several advantages, including, but not limited to increasing the likelihood that the vessel wall is in close proximity to the ring-shaped cutting portion 122 for cutting of the vessel wall and allowing the portion removed to be retained within the recess, for instance, to decrease the likelihood of the portion separating from the gripping device 140 if, for instance, the distal end of the catheter apparatus 100 brushes against a vessel wall, a clip, a surgical instrument, or any other object prior to removing the catheter apparatus 100 from the body of the patient. Separation or dislodgement of the portion from the gripping device 140 could lead to the portion being released into the bloodstream, which could lead to various issues including, for instance, vessel obstruction or blockage.

Referring now to FIG. 5, in some examples, a catheter apparatus 500 is configured to cut through a vessel wall and can be similar to the catheter apparatus 100 described herein. In some examples, the catheter apparatus 500 includes an elongate body similar to the elongate body 102 described herein. In some examples, the elongate body 502 includes a tubular body. The elongate body can include a distal end configured for insertion within the body. In some examples, the distal end includes a widened portion 504 at or near the distal end. In some examples, the widened portion 504 is sized to be at least as wide as the width (or diameter) of the vessel through which the catheter apparatus 500 is intended to be guided. By sizing the widened portion 504 at least as wide as the width of the vessel, the widened portion 504 can function to center the distal end of the catheter apparatus 500 within the vessel to aid in guiding the catheter apparatus 500 through the vessel and positioning it for cutting of the vessel wall. In other examples, the widened portion 504 can be sized to be smaller than the width (or diameter) of the vessel through which the catheter 500 is intended to be guided to allow for greater ease of insertion and guiding of the catheter apparatus 500 within the vessel than if the widened portion 504 were sized as wide or wider than the vessel. In some examples, as described in greater detail herein, the widened portion 504 is sized to correspond with a ring, a clip, or another device used in an anastomosis procedure to inhibit the catheter apparatus 500 from being inserted too far into the vessel to lessen the likelihood of potentially accidentally cutting a portion of a vessel that was not intended to be cut. Although shown as separate from the elongate body, in some examples, the widened portion 504 can be integrally formed with the elongate body.

In some examples, the catheter apparatus 500 includes a cutting device 520 disposed at the distal end of the elongate body. In some examples, the cutting device 520 extends beyond the distal end. In other examples, the cutting device 520 can be flush with or recessed within the distal end. In various examples, the cutting device 520 can extend beyond, be flush with, or be recessed within the widened portion 504. The cutting device 520, in some examples, includes a substantially ring-shaped cutting portion 522 configured to selectively cut a hole in the vessel wall. In some examples, the ring-shaped cutting portion 522 includes a first dimension and a second dimension, wherein the first dimension is greater than the second dimension. In some examples and in a manner similar to that described herein with respect to the widened portion 104 of the catheter apparatus 100, the widened portion 504 of the catheter apparatus 500 is sized to correspond with a ring, a clip, or another device used in an anastomosis procedure to inhibit the catheter apparatus 500 from being inserted too far into the vessel to lessen the likelihood of potentially accidentally cutting through a portion of a vessel that was not intended to be cut through.

In some examples, the catheter apparatus 500 includes a gripping device 540 similar to the gripping device 140 described herein. The gripping device 540 can be configured to grip the vessel wall to maintain the cutting device 520 proximate the vessel wall during cutting of the hole in the vessel wall. In some examples, the gripping device 540 can be disposed within and surrounded by the substantially ring-shaped cutting portion 522. In further examples, the gripping device 540 encompasses substantially the entire area within the substantially ring-shaped cutting portion 522. In other examples, the gripping device 540 can be disposed outside the substantially ring-shaped cutting portion 522. In still further examples, the gripping device 540 can be disposed both within and outside of the substantially ring-shaped cutting portion 522. The gripping device 540, in various examples, is configured to retain a portion of the vessel wall removed during cutting of the vessel wall. That is, with the substantially ring-shaped cutting portion 522, the cutting device 520 cuts or ablates a similarly sized and shaped ring-shaped portion from the vessel wall, which leaves a free portion of the vessel wall (substantially corresponding to the size and shape of the interior of the substantially ring-shaped cutting portion 522) now unattached to the vessel wall, which the gripping device 540 can retain after cutting of the blood vessel and remove from within the body of the patient.

In some examples, the gripping device 540 includes a vacuum device that creates a suction force to grip and retain the portion of the vessel wall. In some examples, the gripping device 540 can include a cover 542 at a distal end 540A of the gripping device 540 to inhibit unwanted objects from being sucked into the end of the gripping device 540. For instance, in some examples, the cover 542 can be configured to inhibit the cut-out portion of the vessel wall from being sucked into the gripping device 540. In some examples, the cover 542 can be configured to hold the vessel wall in a particular position with respect to the cutting device 520 during cutting of the vessel wall. For instance, maintaining the vessel wall in the particular position can, among other things, facilitate proper dimensioning of the hole to be cut by inhibiting the vessel wall from being sucked too far into the gripping device 540 (and potentially making a larger than intended hole in the vessel wall) or moving excessively during cutting (and potentially leading to a hole being formed with improper dimensions).

The cover 542, in some examples, can include one or more holes 542A to allow the suction force of the gripping device 540 to grip the portion of the vessel wall while, at the same time, inhibiting the portion of the vessel wall (or other objects larger than the one or more holes 542A in the cover 542) from being sucked into the distal end 540A of the gripping portion 540. The cover 542 shown in FIG. 5 includes a circular holes 542A. However, in other examples, the cover can include differently configured holes (differently shaped, for instance), a screen, protrusions or abutments extending within the distal end 540A, or other structures or configurations capable of inhibiting objects from being taken in by the gripping device 540. In some examples, the gripping device 540 is recessed with respect to the ring-shaped cutting portion 522, such that, when the gripping device 540 grips the vessel wall, a portion of the vessel wall is drawn within the recess (for instance, to abut the cover 542). Such a configuration allows for several advantages, including, but not limited to increasing the likelihood that the vessel wall is in close proximity to the ring-shaped cutting portion 522 for cutting of the vessel wall and allowing the portion removed to be retained within the recess, for instance, to decrease the likelihood of the portion separating from the gripping device 540 if, for instance, the distal end of the catheter apparatus 500 brushes against a vessel wall, a clip, a surgical instrument, or any other object prior to removing the catheter apparatus 500 from the body of the patient. Separation or dislodgement of the portion from the gripping device 540 could lead to the portion being released into the bloodstream, which could lead to various issues including, for instance, vessel obstruction or blockage.

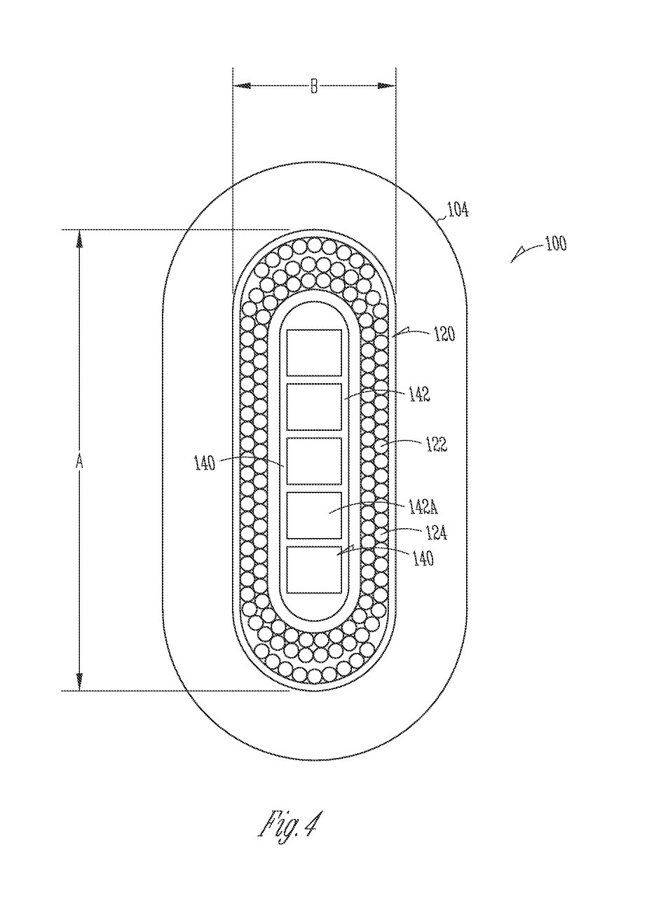

Referring now to FIGS. 1-5, the ring-shaped cutting portion 122, 522 can take different shapes in various examples. For instance, the ring-shaped cutting portion 122 can include an elliptical ring shape with the first dimension being a major axis of the elliptical ring shape and the second dimension being a minor axis of the elliptical ring shape. In further examples, the ring-shaped cutting portion 122 includes a flattened elliptical ring shape or a “racetrack” shape, as shown in FIGS. 1-4. As shown in FIG. 4, the flattened elliptical ring-shaped cutting portion 122 can include a first dimension A and a second dimension B, with the first dimension A being greater than the second dimension B. In other examples, as shown in FIG. 5, the substantially ring-shaped cutting portion 522 of the cutting device 520 can include a rectangular ring shape, with a first dimension including a length L and a second dimension including a width W. In still further examples, other shapes for the substantially ring-shaped cutting portion are contemplated, such as circular, square-shaped, diamond-shaped, triangular, hexagonal (or any polygonal shape), or the like.

Referring specifically to FIGS. 4 and 5, in some examples, the size of the ring-shaped cutting portion 122, 522 can be determined according to the vessel or vessels to be cut using the ring-shaped cutting portion 122, 522. That is, the first dimension A, L and/or the second dimension B, W can be chosen to correspond to the size of one or more of the vessels that are to be cut using the cutting device 120, 520. In some examples, sizing of the substantially ring-shaped cutting portion 122, 522 includes sizing the first dimension A, L to correspond to a size of a first vessel to be cut with the cutting device 120, 520. In some examples, sizing of the substantially ring-shaped cutting portion 122, 522 includes sizing the second dimension B, W to correspond to a size of a second vessel to be cut with the cutting device 120, 520. For instance, the first dimension or the major axis A of the flattened elliptical ring-shaped cutting portion 122 can be chosen to correspond to a flow surface of the largest recipient vessel to be cut using the cutting device 120, and/or the second dimension or the minor axis B of the flattened elliptical ring-shaped cutting portion 122 can be chosen to correspond to a diameter of the smallest recipient vessel to be cut using the cutting device 120. In another example, the first dimension or the length L of the rectangular ring-shaped cutting portion 522 can be chosen to correspond to a flow surface of the largest recipient vessel to be cut using the cutting device 520, and/or the second dimension or the width W of the rectangular ring-shaped cutting portion 522 can be chosen to correspond to a diameter of the smallest recipient vessel to be cut using the cutting device 520. In some examples, the first and second dimensions A, L, B, W the ring-shaped cutting portion 122, 522 can be sized and shaped to match a surface area of an anastomosis to a surface area of the recipient vessel, such that the flow surface area of the recipient vessel and the anastomosis surface area is substantially equal to or larger than the flow surface area of the recipient vessel. For instance, the first and second dimensions A, L, B, W the ring-shaped cutting portion 122, 522 can be sized and shaped to allow cutting of a hole in a vessel that is substantially equal to (or larger than) the flow surface of the recipient vessel. In this way, stenosis in the recipient vessel can be inhibited. In other examples, the size of one or more of the first and second dimensions A, B, L, W can be sized according to other aspects or dimensions of the one or more vessels to be cut with the cutting device 120, 520.

Referring again to FIGS. 1-5, the cutting device 120, 520, in various examples, can include different forms of cutting mechanisms. In some examples, the cutting device 120, 520 can include a laser cutting device 120, 520 configured to cut through the vessel wall. In some examples, the laser cutting device 120, 520 includes a plurality of optical fibers 124, 524 each including an end 124A, 524A. The ends 124A, 524A of the optical fibers 124, 524, in some examples, are arranged to define the substantially ring-shaped cutting portion 122, 522. For instance, with respect to the example shown in FIGS. 1-4, the ends 124A of the optical fibers 124 are arranged to define a substantially planar flattened ellipse to form the flattened elliptical ring-shaped cutting portion 122. With respect to the example shown in FIG. 5, the ends 524A of the optical fibers 524 are arranged to define a substantially planar rectangle to form the rectangular ring-shaped cutting portion 522. In some examples, the substantially ring-shaped cutting portion 122, 522 is configured to emit a laser distally from the catheter apparatus 100, 500.

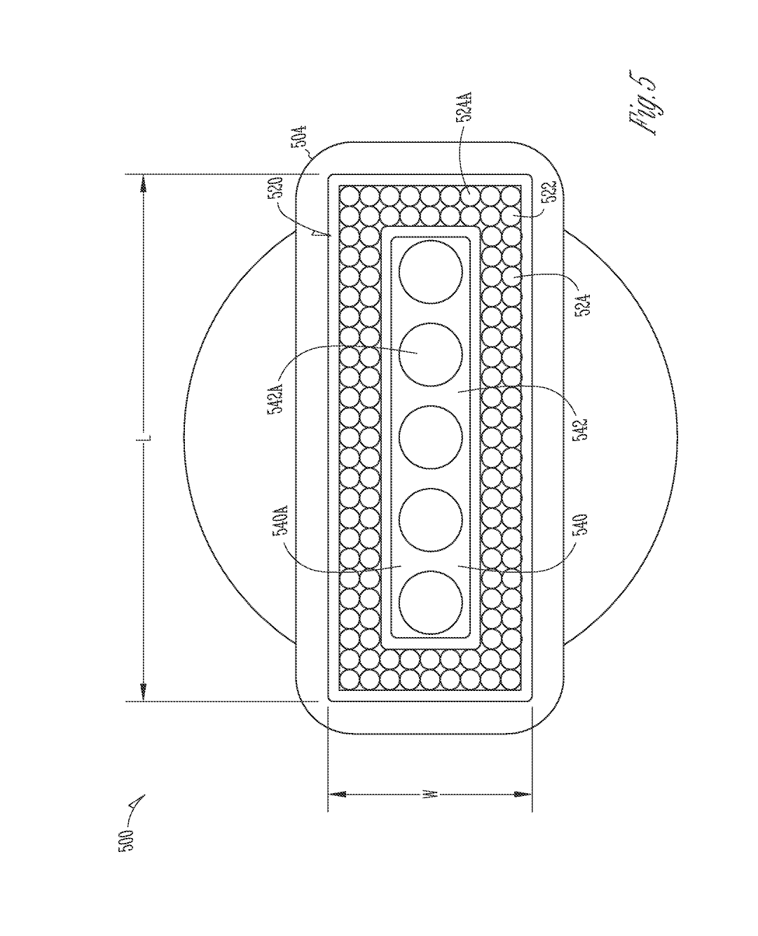

Referring now to FIG. 6, an example of the catheter apparatus 100 is shown. Although shown and described with respect to the catheter apparatus 100, it should be understood that the description of FIG. 6 can apply to at least some examples of the catheter apparatus 500, as shown and described herein. In some examples, the cutting device 120 can include a laser generator 126 configured to produce the laser energy to be emitted by the ends 124A of the optical fibers 124. In some examples, a proximal end of each optical fiber 124 is coupled to the laser generator 126 with the optical fibers 124 running from the laser generator 126, through the elongate body 102, and terminating at the substantially ring-shaped cutting portion 122. In this way, the laser generator 126 can produce a laser which can propagate through the optical fibers 124 and be emitted from the ends 124A of the optical fibers 124. In some examples, the laser is emitted from the ends 124A of the optical fibers 124 which are arranged to form the ring-shaped cutting portion 122 in order to cut or ablate a hole in a vessel wall or other structure. In some examples, various other types of cutting devices 120 are contemplated, including, but not limited to, a scalpel, a punch, a bi/mono polar cutting device, a radiofrequency cutting device, or an ultrasonic cutting device.

In some examples, the optical fibers 124 are disposed immediately within the elongate body 102. The optical fibers 124 can be arranged within the elongate body 102 in various ways, including, but not limited to a continuous ring, a segmented ring, or a single bundle of optical fibers. In some examples, the catheter apparatus 100 includes an inner tube 106, for instance disposed within the optical fibers 124. The inner tube 106 can provide additional structure to the catheter apparatus 100. The inner tube 106, in some examples, can define, or at least partially define, an inner lumen 144 of the catheter apparatus 100.

In some examples, the catheter apparatus 100 includes an end portion 108 disposed at the distal end 102A of the elongate body 102. The end portion 108 can provide, in some examples, a relatively rigid structure for the end of the catheter apparatus 100. In some examples, the end portion 108 partially defines the inner lumen 144 and provides a nozzle 144A for the vacuum gripping device 140 disposed at the distal end 102A of the elongate body 102. In some examples, the gripping device 140 is configured to create a suction force between the nozzle 144A and the vessel wall to grip the vessel wall. The vacuum gripping device 140 includes a vacuum source 146 coupled to the nozzle 144A. In some examples, the vacuum source 146 is coupled to a proximal portion of the inner lumen 144 of the catheter apparatus 100 to fluidly couple the vacuum source 146 with the nozzle 144A to allow the suction force to be created at the nozzle 144A with operation of the vacuum source. In this way, operation of the vacuum source 146 allows the gripping device 140 to selectively grip the vessel wall to be cut and also to retain the portion of the vessel wall removed after cutting. The nozzle 144A, in some examples, is disposed within and surrounded by the substantially ring-shaped cutting portion 122 of the cutting device 120. In some examples, the nozzle 144A can be configured to inhibit the cut-out portion of the vessel wall or unwanted objects from being sucked into the end of the gripping device 140 without requiring the cover 142 to be used (although it should be noted that the cover 142 can be used even with the nozzle 144A being configured to inhibit the cut-out portion of the vessel wall or unwanted objects from being sucked into the end of the gripping device 140). For instance, an interior surface of the nozzle 144A can be substantially conically-shaped to inhibit the cut-out portion of the vessel wall or unwanted objects from being drawn too far into the end of the gripping device 140. In further examples, the interior surface of the nozzle 144A can include other shapes to inhibit the cut-out portion or other objects from being drawn too far into the end of the gripping device 140.

In some examples, the cover 142, although shown as being substantially flat in FIG. 6, can be differently shaped. For instance, the cover 142, when viewed in cross section, can be slanting or sloping, substantially pyramidal, substantially conical, substantially spherical, wave-shaped, or zig-zag-shaped. In some examples, the cover 142 can be attached to the end portion 108. For instance, the cover 142 can be integrally formed with the end portion 108. In some examples, the cover 142 is separately formed but attached to the end portion 108 in some manner including, but not limited to a snap fit, a friction fit, an adhesive, welding (for instance, ultrasonic welding), a threaded connection, or some other manner of attachment.

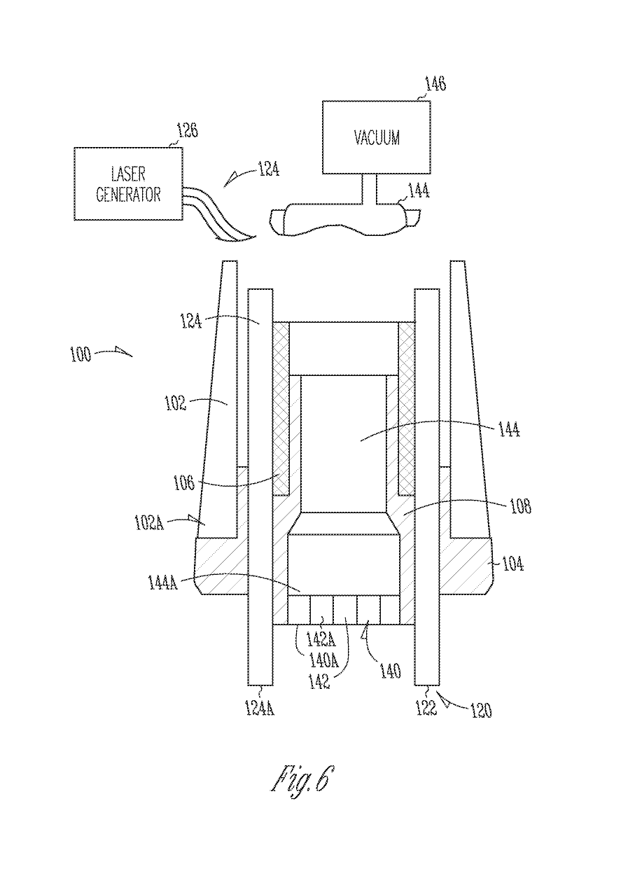

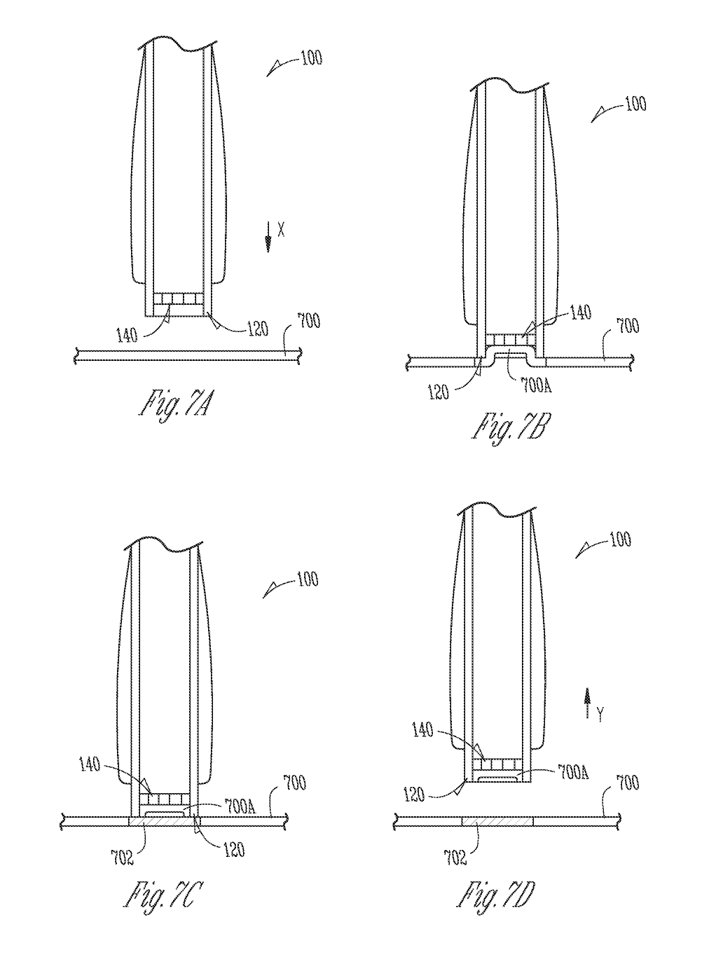

FIGS. 7A-7D show a simplified depiction of the catheter apparatus 100 cutting through a vessel wall 700. Although shown and described with respect to the catheter apparatus 100, it should be understood that the description of FIGS. 7A-7D can apply to at least some examples of the catheter apparatus 500, as shown and described herein.

Referring to FIGS. 7A-7D, the catheter apparatus 100 can be used to cut through the vessel wall 700 in the context of anastomosis, for instance. It is noted that the depiction is simplified at least in part because the catheter apparatus 100 is shown merely with the vessel wall 700 to be cut. That is, the catheter apparatus 100 is not shown disposed within a vessel. In at least some example anastomosis procedures, the catheter apparatus 100 would be disposed within a vessel for cutting of the vessel wall 700. Also, only one vessel wall 700 is shown and no other vessel or vessel wall is shown attached to the vessel wall 700. In at least some example anastomosis procedures, the vessel wall 700 would be attached (for instance, clipped, sutured, or otherwise affixed) to a portion (for instance, a vessel wall or an end of another vessel) during cutting of the vessel wall 700.

In some examples, in use, the catheter apparatus 100 is advanced along arrow X toward the vessel wall 700 to be cut, as shown in FIG. 7A. Once positioned proximate the vessel wall 700, the gripping device 140 can be used to grip the vessel wall, as shown in FIG. 71B. In some examples, the gripping device 140 actually draws in a portion 700A of the vessel wall 700 into the recess of the gripping device 140 with respect to the cutting device 120. In some examples, gripping the vessel wall 700 includes creating the suction force between the nozzle 144 (see FIG. 6) of the gripping device 140 and the vessel wall 700. In further examples, the gripping device 140 includes a vacuum gripping device 140 for creating the suction force to grip the vessel wall.

As shown in FIG. 7C, the vessel wall 700 can then be cut using the cutting device 120 disposed at the distal end of the catheter apparatus 100. In some examples, the cutting device 120 can include the substantially ring-shaped cutting portion 122 similar to that described herein in which the first dimension is greater than the second dimension of the substantially ring-shaped cutting portion 122 (see FIG. 4). The gripping device 140 can be disposed within and surrounded by the substantially ring-shaped cutting portion 122 of the cutting device 120. The cutting device 120 can be configured to cut the vessel wall 700 along the substantially ring-shaped cutting portion 122 to form a hole 702 in the vessel wall 700. In some examples, cutting the vessel wall 700 includes emitting a laser distally from the substantially ring-shaped cutting portion 122, with the laser being configured to cut through or ablate the vessel wall 700. As shown in FIG. 71), once a portion 700A is cut from the vessel wall 700, the portion 700A can be retained by the catheter apparatus 100 using the gripping device 140. In this way, the portion 700A of the vessel wall 700 can be removed with movement of the catheter apparatus 100 in the direction of arrow Y and removal of the catheter apparatus 100 from the body of the patient.

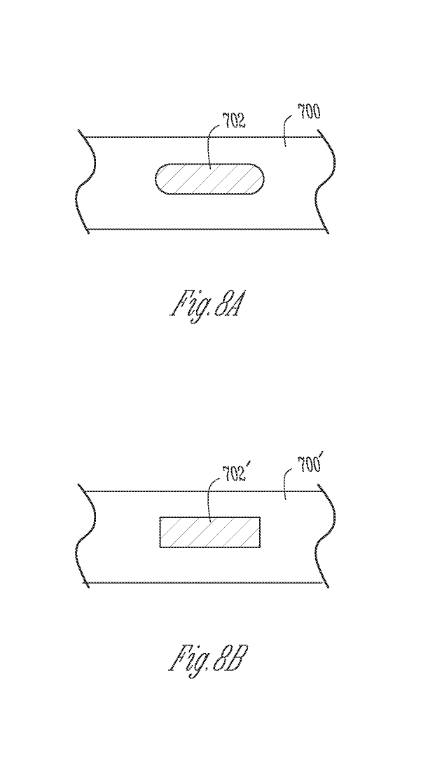

Depending upon the shape of the cutting portion of the cutting device, variously shaped holes can be formed in the vessel wall using the catheter apparatuses 100, 500 described herein and used, for instance, as shown in FIGS. 7A-7D. For instance, referring to FIGS. 4 and 8A, using the catheter apparatus 100 including the substantially flattened elliptical ring-shaped cutting portion 122, a substantially flattened elliptical hole 702 can be formed in the vessel wall 700, with dimensions of the hole 702 substantially corresponding to the dimensions (the major axis A and the minor axis B) of the substantially flattened elliptical ring-shaped cutting portion 122. In another example, referring to FIGS. 5 and 8B, using the catheter apparatus 500 including the rectangular ring-shaped cutting portion 522, a rectangular hole 702′ can be formed in a vessel wall 700′, with dimensions of the hole 702′ substantially corresponding to the dimensions (the length L and the width W) of the rectangular ring-shaped cutting portion 522. In other examples, cutting portions including different shapes are contemplated herein.

Referring again to FIGS. 1-5, the example catheter apparatuses described herein are advantageous in several respects. For instance, while previous catheter shapes were prone to creating stenosis in a recipient artery, the shapes of the substantially ring-shaped cutting portions 122, 522 allow for the creation of a flow surface (a hole in a vessel wall) that is substantially equal to (if not larger than) the flow surface of the recipient vessel so that the likelihood of creating a stenosis in the recipient vessel is lessened. Moreover, in some examples, the shapes of the substantially ring-shaped cutting portions 122, 522 (and the corresponding gripping devices 140, 540) are configured to facilitate maneuvering of the catheter apparatuses 100, 500 within the desired vessels and successful cutting of the vessel wall and capturing of the portion removed from the vessel wall. That is, the elliptical or flattened elliptical ring-shaped cutting portion 122, with the major axis A being greater than the minor axis B, and the rectangular ring-shaped cutting portion 522, with the length L being greater than the width W, enable movement of the respective catheter apparatuses 100, 500 through the desired vessels by sizing the dimensions A, B, L, W of the ring-shaped cutting portions 122, 522 to correspond with the vessels to be cut. Also, the shape of the ring-shaped cutting portions 122, 522 (and the corresponding shapes of the gripping portions 140, 540), with the first dimensions A, L being greater than the second dimensions B, W, allows for an increased cutting and gripping surface area for each catheter apparatus 100, 500 in relation to the size constraints of the vessels with which the catheter apparatuses 100, 500 are to be used. Because of the increased surface areas of the ring-shaped cutting portions 122, 522 and the gripping devices 140, 540, the likelihood of a clean cut by the ring-shaped cutting portions 122, 522 is increased and the gripping of the vessel wall, initially, and retention of the removed portion is facilitated. Although described with respect to substantially elliptical and rectangular ring-shaped cutting portions 122, 522 (and corresponding gripping devices 140, 540), it should be understood that other shapes optimizing the available cutting and gripping surface areas are contemplated herein.

### Additional Notes and Examples

Example 1 can include subject matter (such as an apparatus, a system, a method, a means for performing acts, or a machine-readable medium including instructions that, when performed by the machine, cause the machine to perform acts) that can comprise a catheter apparatus configured to cut through a vessel wall. The catheter apparatus can comprise: an elongate body including a distal end; a cutting device disposed at the distal end of the elongate body, the cutting device including a substantially ring-shaped cutting portion including a first dimension and a second dimension, wherein the first dimension is greater than the second dimension, wherein the cutting device is configured to selectively cut a hole in the vessel wall; and a gripping device disposed at the distal end of the elongate body, the gripping device configured to grip the vessel wall to maintain the cutting device proximate the vessel wall during cutting of the hole in the vessel wall, the gripping device further configured to retain a portion of the vessel wall removed during cutting of the vessel wall.

Example 2 can include or use, or can optionally be combined with the subject matter of Example 1 to include or use a catheter apparatus, wherein the cutting device includes a laser cutting device configured to cut through the vessel wall.

Example 3 can include or use, or can optionally be combined with the subject matter of Example 2 to include or use a catheter apparatus, wherein the laser cutting device includes a plurality of optical fibers each including an end, the ends of the optical fibers arranged to define the substantially ring-shaped cutting portion.

Example 4 can include or use, or can optionally be combined with the subject matter of Examples 1-3 to include or use a catheter apparatus, wherein the substantially ring-shaped cutting portion is configured to emit a laser distally from the catheter apparatus.

Example 5 can include or use, or can optionally be combined with the subject matter of Examples 1-4 to include or use a catheter apparatus, wherein the substantially ring-shaped cutting portion includes an elliptical ring shape, wherein the first dimension includes a major axis and the second dimension includes a minor axis.

Example 6 can include or use, or can optionally be combined with the subject matter of Example 4 to include or use a catheter apparatus, wherein the elliptical ring shape of the substantially ring-shaped cutting portion includes a flattened elliptical ring shape.

Example 7 can include or use, or can optionally be combined with the subject matter of Examples 1-6 to include or use a catheter apparatus, wherein the substantially ring-shaped cutting portion includes a rectangular ring shape, wherein the first dimension includes a length and the second dimension includes a width.

Example 8 can include or use, or can optionally be combined with the subject matter of Examples 1-7 to include or use a catheter apparatus, wherein the gripping device includes a vacuum device including a nozzle disposed at the distal end of the elongate body, the vacuum device configured to create a suction force between the nozzle and the vessel wall to grip the vessel wall.

Example 9 can include or use, or can optionally be combined with the subject matter of Example 8 to include or use a catheter apparatus, wherein the nozzle is disposed within and surrounded by the substantially ring-shaped cutting portion of the cutting device.

Example 10 can include or use, or can optionally be combined with the subject matter of Example 8 to include or use a catheter apparatus, wherein the vacuum device includes a vacuum source coupled to the nozzle.

Example 11 can include or use, or can optionally be combined with the subject matter of Examples 1-10 to include or use a catheter apparatus, wherein the elongate body includes a tubular body.

Example 12 can include, or can be combined with the subject matter of one or any combination of Examples 1-11 to optionally include, subject matter (such as an apparatus, a method, a means for performing acts, or a machine-readable medium including instructions that, when performed by the machine, cause the machine to perform acts) that can comprise a catheter apparatus configured to cut through a vessel wall. The catheter apparatus can comprise: an elongate body including a distal end; a laser cutting device disposed at the distal end of the elongate body, the laser cutting device including a substantially elliptical ring-shaped cutting portion, including a major axis and a minor axis, wherein the cutting device is configured to selectively cut a hole in the vessel wall; and a vacuum device including a nozzle disposed at the distal end of the elongate body, the nozzle is disposed within and surrounded by the substantially elliptical ring-shaped cutting portion of the laser cutting device, the vacuum device configured to create a suction force between the nozzle and the vessel wall to grip the vessel wall and to maintain the laser cutting device proximate the vessel wall during cutting of the hole in the vessel wall, the vacuum device further configured to retain a portion of the vessel wall removed during cutting of the vessel wall.

Example 13 can include or use, or can optionally be combined with the subject matter of Example 12 to include or use a catheter apparatus, wherein the laser cutting device includes a plurality of optical fibers each including an end, the ends of the optical fibers arranged to define the substantially elliptical ring-shaped cutting portion.

Example 14 can include or use, or can optionally be combined with the subject matter of Examples 12-13 to include or use a catheter apparatus, wherein the substantially elliptical ring-shaped cutting portion includes a flattened elliptical ring shape.

Example 15 can include or use, or can optionally be combined with the subject matter of Examples 12-14 to include or use a catheter apparatus, wherein the substantially elliptical ring-shaped cutting portion is configured to emit a laser distally from the catheter apparatus.

Example 16 can include, or can be combined with the subject matter of one or any combination of Examples 1-15 to optionally include, subject matter (such as an apparatus, a method, a means for performing acts, or a machine-readable medium including instructions that, when performed by the machine, cause the machine to perform acts) that can comprise a method of cutting through a vessel wall. The method can comprise: gripping the vessel wall using a gripping device disposed at a distal end of a catheter apparatus; cutting the vessel wall using a cutting device disposed at the distal end of the catheter apparatus, the cutting device including a substantially ring-shaped cutting portion including a first dimension and a second dimension, wherein the first dimension is greater than the second dimension, the gripping device being disposed within and surrounded by the substantially ring-shaped cutting portion of the cutting device, the cutting device cutting the vessel wall along the substantially ring-shaped cutting portion to form a hole in the vessel wall; and retaining a portion of the vessel wall removed during cutting of the hole in the vessel wall, the portion of the vessel wall retained using the gripping device.

Example 17 can include or use, or can optionally be combined with the subject matter of Example 16 to include or use a method, wherein cutting the vessel wall includes emitting a laser distally from the substantially ring-shaped cutting portion, the laser being configured to cut through the vessel wall.

Example 18 can include or use, or can optionally be combined with the subject matter of Examples 16-17 to include or use a method, wherein the cutting device includes a substantially flattened elliptical ring-shaped cutting portion configured to cut a substantially flattened elliptical hole in the vessel wall, wherein the first dimension includes a major axis and the second dimension includes a minor axis.

Example 19 can include or use, or can optionally be combined with the subject matter of Examples 16-18 to include or use a method, wherein the cutting device includes a rectangular ring-shaped cutting portion configured to cut a rectangular hole in the vessel wall, wherein the first dimension includes a length and the second dimension includes a width.

Example 20 can include or use, or can optionally be combined with the subject matter of Examples 16-19 to include or use a method, wherein gripping the vessel wall includes creating a suction force between a nozzle of the gripping device and the vessel wall, the gripping device including a vacuum device.

Example 21 can include or use, or can optionally be combined with the subject matter of Examples 16-20 to include or use a method, including sizing the substantially ring-shaped cutting portion of the cutting device to correspond to a vessel to be cut.

Example 22 can include or use, or can optionally be combined with the subject matter of Example 21 to include or use a method, wherein sizing the substantially ring-shaped cutting portion includes sizing the first dimension to correspond to a size of a first vessel to be cut with the cutting device.

Example 23 can include or use, or can optionally be combined with the subject matter of Example 22 to include or use a method, wherein sizing the substantially ring-shaped cutting portion includes sizing the second dimension to correspond to a size of a second vessel to be cut with the cutting device.

Example 24 can include or use, or can optionally be combined with the subject matter of Examples 21-23 to include or use a method, wherein sizing the substantially ring-shaped cutting portion includes sizing the second dimension to correspond to a size of a second vessel to be cut with the cutting device.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A catheter apparatus configured to cut through a vessel wall, the catheter apparatus comprising:
an elongate body including a distal end;
a cutting device disposed at the distal end of the elongate body, the cutting device including a substantially ring-shaped cutting portion including a first dimension and a second dimension, wherein the first dimension is greater than the second dimension, wherein the cutting device is configured to selectively cut a hole in the vessel wall; and
a gripping device disposed at the distal end of the elongate body, the gripping device configured to grip the vessel wall to maintain the cutting device proximate the vessel wall during cutting of the hole in the vessel wall, the gripping device further configured to retain a portion of the vessel wall removed during cutting of the vessel wall.

2. The catheter apparatus of claim 1, wherein the cutting device includes a laser cutting device configured to cut through the vessel wall.

3. The catheter apparatus of claim 2, wherein the laser cutting device includes a plurality of optical fibers each including an end, the ends of the optical fibers arranged to define the substantially ring-shaped cutting portion.

4. The catheter apparatus of claim 1, wherein the substantially ring-shaped cutting portion is configured to emit a laser distally from the catheter apparatus.

5. The catheter apparatus of claim 1, wherein the substantially ring-shaped cutting portion includes an elliptical ring shape, wherein the first dimension includes a major axis and the second dimension includes a minor axis.

6. The catheter apparatus of claim 5, wherein the elliptical ring shape of the substantially ring-shaped cutting portion includes a flattened elliptical ring shape.

7. The catheter apparatus of claim 1, wherein the substantially ring-shaped cutting portion includes a rectangular ring shape, wherein the first dimension includes a length and the second dimension includes a width.

8. The catheter apparatus of claim 1, wherein the gripping device includes a vacuum device including a nozzle disposed at the distal end of the elongate body, the vacuum device configured to create a suction force between the nozzle and the vessel wall to grip the vessel wall.

9. The catheter apparatus of claim 8, wherein the nozzle is disposed within and surrounded by the substantially ring-shaped cutting portion of the cutting device.

10. The catheter apparatus of claim 8, wherein the vacuum device includes a vacuum source coupled to the nozzle.

11. The catheter apparatus of claim 1, wherein the elongate body includes a tubular body.

12. A catheter apparatus configured to cut through a vessel wall, the catheter apparatus comprising:
an elongate body including a distal end;
a laser cutting device disposed at the distal end of the elongate body, the laser cutting device including a substantially elliptical ring-shaped cutting portion, including a major axis and a minor axis, wherein the cutting device is configured to selectively cut a hole in the vessel wall; and
a vacuum device including a nozzle disposed at the distal end of the elongate body, the nozzle is disposed within and surrounded by the substantially elliptical ring-shaped cutting portion of the laser cutting device, the vacuum device configured to create a suction force between the nozzle and the vessel wall to grip the vessel wall and to maintain the laser cutting device proximate the vessel wall during cutting of the hole in the vessel wall, the vacuum device further configured to retain a portion of the vessel wall removed during cutting of the vessel wall.

13. The catheter apparatus of claim 12, wherein the laser cutting device includes a plurality of optical fibers each including an end, the ends of the optical fibers arranged to define the substantially elliptical ring-shaped cutting portion.

14. The catheter apparatus of claim 12, wherein the substantially elliptical ring-shaped cutting portion includes a flattened elliptical ring shape.

15. The catheter apparatus of claim 12, wherein the substantially elliptical ring-shaped cutting portion is configured to emit a laser distally from the catheter apparatus.

16. A method of cutting through a vessel wall, the method comprising:
gripping the vessel wall using a gripping device disposed at a distal end of a catheter apparatus;
cutting the vessel wall using a cutting device disposed at the distal end of the catheter apparatus, the cutting device including a substantially ring-shaped cutting portion including a first dimension and a second dimension, wherein the first dimension is greater than the second dimension, the gripping device being disposed within and surrounded by the substantially ring-shaped cutting portion of the cutting device, the cutting device cutting the vessel wall along the substantially ring-shaped cutting portion to form a hole in the vessel wall; and
retaining a portion of the vessel wall removed during cutting of the hole in the vessel wall, the portion of the vessel wall retained using the gripping device.

17. The method of claim 16, wherein cutting the vessel wall includes emitting a laser distally from the substantially ring-shaped cutting portion, the laser being configured to cut through the vessel wall.

18. The method of claim 16, wherein the cutting device includes a substantially flattened elliptical ring-shaped cutting portion configured to cut a substantially flattened elliptical hole in the vessel wall, wherein the first dimension includes a major axis and the second dimension includes a minor axis.

19. The method of claim 16, wherein the cutting device includes a rectangular ring-shaped cutting portion configured to cut a rectangular hole in the vessel wall, wherein the first dimension includes a length and the second dimension includes a width.

20. The method of claim 16, wherein gripping the vessel wall includes creating a suction force between a nozzle of the gripping device and the vessel wall, the gripping device including a vacuum device.

21. The method of claim 16, comprising sizing the substantially ring-shaped cutting portion of the cutting device to correspond to a vessel to be cut.

22. The method of claim 21, wherein sizing the substantially ring-shaped cutting portion includes sizing the first dimension to correspond to a size of a first vessel to be cut with the cutting device.

23. The method of claim 22, wherein sizing the substantially ring-shaped cutting portion includes sizing the second dimension to correspond to a size of a second vessel to be cut with the cutting device.

24. The method of claim 21, wherein sizing the substantially ring-shaped cutting portion includes sizing the second dimension to correspond to a size of a second vessel to be cut with the cutting device.
